(19) United States
(12) Patent Application Publication
(10) Pub. No.: US 2017/0079857 A1
(43) Pub. Date: Mar. 23, 2017

(54) ABSORBENT ARTICLES HAVING CHANNELS, STRETCHABLE WAISTBANDS AND BACK EARS

(71) Applicant: The Procter & Gamble Company, Cincinnati, OH (US)

(72) Inventors: Keith Richard WILLHAUS, Cincinnati, OH (US); Erneston Gabriel Bianchi, Oberursel (DE); Paul Thomas Weisman, Cincinnati, OH (US)

(21) Appl. No.: 15/272,469

(22) Filed: Sep. 22, 2016

Related U.S. Application Data
(60) Provisional application No. 62/221,867, filed on Sep. 22, 2015.

(51) Int. Cl. A61F 13/537; A61F 13/56; A61F 13/551; A61F 13/42; A61F 13/49

(52) U.S. Cl. CPC A61F 13/53713; A61F 13/42; A61F 13/49; A61F 13/55115; A61F 13/5633; A61F 2013/427; A61F 2013/428; A61F 2013/49049; A61F 2013/49093; A61F 2013/530189

(57) ABSTRACT

An absorbent article includes a topsheet, a backsheet, an absorbent core and at least one longitudinally-extending channel that is substantially free of absorbent material. The article further includes a stretchable back waistband extending transversally adjacent the back edge of the article and a pair of stretchable back ears. The back ears each include a fastening tab that can be releasably engaged with a landing zone on the garment-facing side of the article.

### FIELD OF THE INVENTION

The invention is directed to disposable absorbent articles for personal hygiene such as baby diapers, training pants and adult incontinence products.

### BACKGROUND OF THE INVENTION

Absorbent articles for personal hygiene of the type indicated above are designed to absorb and contain body exudates, in particular large quantity of urine. These absorbent articles comprise several layers, typically a topsheet, a backsheet and in-between an absorbent core, among other layers. Absorbent articles should absorb and retain the exudates for a prolonged amount of time, minimize re-wet to keep the wearer dry and avoid soiling of clothes or bed sheets. Various improvements have been suggested in the past to improve fluid acquisition, reduce leakage and improve comfort.

Absorbent cores comprising longitudinally-extending material-free channels have been suggested in the art. The channels may help directing an incoming fluid quickly across a larger area of the core. When the top side of the core wrap is bonded to its bottom side through these material-free areas, the longitudinal structural rigidity of the article through the crotch region of the article can be improved in the wet state, due to the swelling of the wetted absorbent polymer particles.

However, it has been discovered that this structural rigidity may have undesirable effects on appearance, fit and and/or comfort. In particular, the longitudinal structural rigidity can cause the back region of the absorbent core to tend to bulge outwardly from the wearer's body in bulky protrusion that can create an unsightly appearance and adversely impact comfort.

There is a need for continuously improving the fluid handling properties (acquisition, distribution and retention) and the wearing comfort of absorbent articles in wet and dry state, while keeping the unit costs as low as possible. The present invention addresses these needs.

### SUMMARY OF THE INVENTION

The present invention is for absorbent articles in particular taped diapers, as indicated in the claims. The absorbent articles of the invention comprise a topsheet, a backsheet, an absorbent core comprising at least one and in particular a pair of longitudinally-extending channels substantially free of absorbent material. The articles further comprise a stretchable waistband adjacent to their back edges and two stretchable back ears. Each back ear comprises a fastening tab that can be releasably engaged with a landing zone disposed towards the front of the garment-facing side of the article.

### BRIEF DESCRIPTION OF THE DRAWINGS

FIG. 1 is a schematic top view of an exemplary absorbent article of the invention in the form a taped diaper presented in an open and flattened-out configuration with the wearer-facing side facing up;

FIG. 2 is a schematic exploded perspective view of the diaper of FIG. 1 showing the main components of the diapers, excluding glues and some elastics for clarity;

FIG. 3 is a schematic cross-sectional view of the diaper of FIG. 1;

FIG. 3a is a schematic close-up view of 3 dimensional wearer-facing material;

FIG. 4 is a top view of the absorbent core of the diaper of FIG. 1 shown in isolation;

FIG. 5a is a cross-sectional view of the absorbent core with the core wrap made of a single substrate;

FIG. 5b is a cross-sectional view of the absorbent core with the core wrap comprising two substrates;

FIG. 6 is a schematic view of a package of absorbent articles of the present invention.

### DETAILED DESCRIPTION OF THE INVENTION

### Introduction

Any preferred or exemplary embodiments described below are not limiting the scope of the claims, unless specifically indicated to do so. The words “typically”, “normally”, “preferably”, “advantageously”, “in particular” and the likes also qualify features which are not intended to limit the scope of the claims unless specifically indicated to do so.

As used herein, the term “wearer” refers to an incontinent person, which may be an adult, a child, or a baby, and that will wear the absorbent article. The term “user” refers to the caregiver that applies the absorbent article on the wearer. The user may be a parent, a family member in general, a professionally employed caregiver or the wearer him/herself.

The term “nonwoven” is used herein in the usual sense in the art and means a manufactured sheet, web or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling, whether or not additionally needled. The fibers may be of natural or man-made origin and may be staple or continuous filaments or be formed in situ. Commercially available fibers have diameters ranging from less than about 0.001 mm to more than about 0.2 mm and they come in several different forms such as short fibers (known as staple, or chopped), continuous single fibers (filaments or monofilaments), untwisted bundles of continuous filaments (tow), and twisted bundles of continuous filaments (yam). Nonwoven webs can be formed by many processes such as meltblowing, spunbonding, solvent spinning, electrospinning, carding and airlaying. The basis weight of nonwoven webs is usually expressed in grams per square meter (g/m² or gsm).

The invention will now be further illustrated with reference to the embodiments as described in the Figures. For ease of discussion, absorbent articles and their components such as the absorbent core will be discussed with reference to the numerals referred to in these Figures. However it should be understood that these exemplary embodiments and the numerals are not intended to limit the scope of the claims, unless specifically indicated. Nothing in this description should be however considered limiting the scope of the claims unless explicitly indicated otherwise. Dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

### General Description of the Article 20

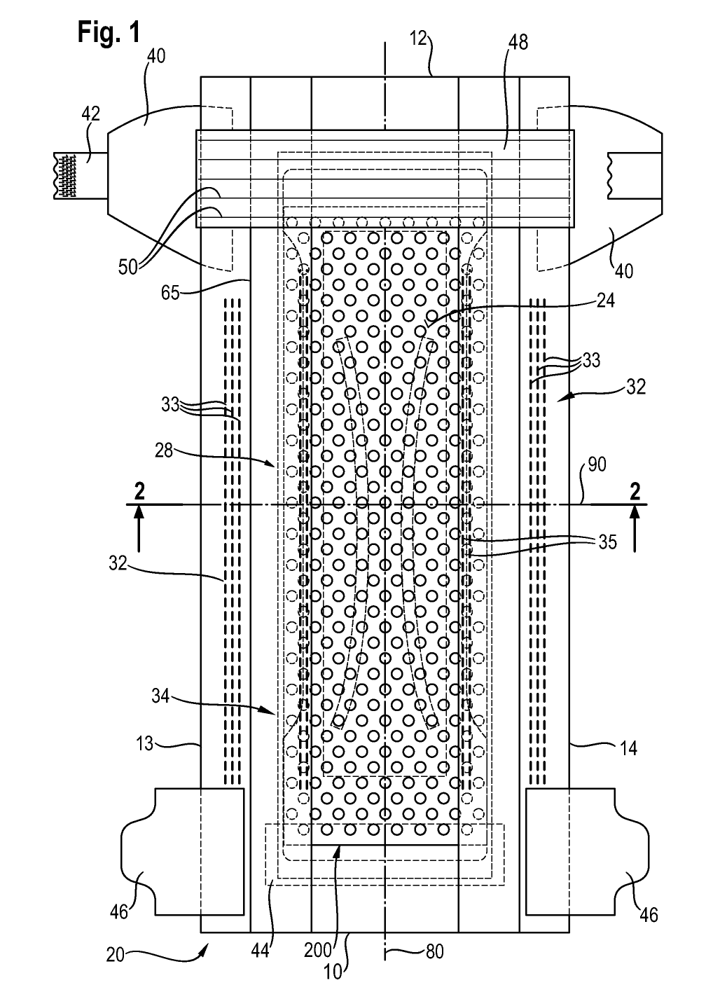

As used herein, the term “absorbent articles” refers to disposable products for personal hygiene which are placed against or in proximity to the body of the wearer to absorb and contain exudates discharged from the body, in particular urine. The absorbent article will now be generally discussed and further illustrated in the form of a baby taped diaper 20 as exemplarily represented in FIG. 1. The diaper is illustrated in a flattened-out configuration with the back ears 40 and the front ears 46 opened and the wearer-facing side turned up.

The absorbent article has a front edge 10, a back edge 12 and the longitudinally-extending side edges 13, 14. The front edge 10 is placed in use towards the front of the wearer and the back edge 12 forms towards the back of the wearer. When the diaper is closed, the front and back edges form together the waist opening for the wearer. Each side edge forms one of the leg openings. The article has a longitudinal direction and a transverse direction defined by the longitudinal axis 80 and transversal axis 90 respectively. The longitudinal axis 80 extends through the middle of the front and back edges 10, 12 of the article, and thus virtually divides the article in symmetrical left side and right side. The article has a length L along the longitudinal axis 80 between the front and back edges of the article. The transversal axis 90 extends perpendicularly to the longitudinal axis and crosses the longitudinal axis at a position halfway between the front edge and the back edge (L/2 from the front and back edges). The transversal axis thus virtually defines a front half and a back half of the absorbent article.

The wearer-facing side of the article typically comprises the topsheet 24, which may be a nonwoven material (or a formed film), and optionally a three-dimensional material that may replace the topsheet completely or may be, as represented, an additional material 200 present on a portion of the topsheet to provide special properties to a portion of the topsheet. A backsheet 25 forms the opposite, garment-facing side of the diaper. Typically the backsheet comprises a liquid-impermeable film, which may be doubled externally by a softer non-woven layer on its surface. The backsheet film may comprise micro-pores to make the film vapor-permeable. Examples of topsheet and backsheet will be further discussed below.

The absorbent articles of the invention may further comprises an acquisition layer 54 (also called distributional layer, acquisition-distribution layer, or secondary-topsheet) placed between the topsheet and the absorbent core. The acquisition layer may in particular be an air-through bonded carded nonwoven. The acquisition layer may be about as wide (in transverse direction) and shorter (in longitudinal direction) as the absorbent core underneath, but other dimensions are possible. The acquisition layer typically does not comprise SAP as this may slow the acquisition and distribution of the fluid.

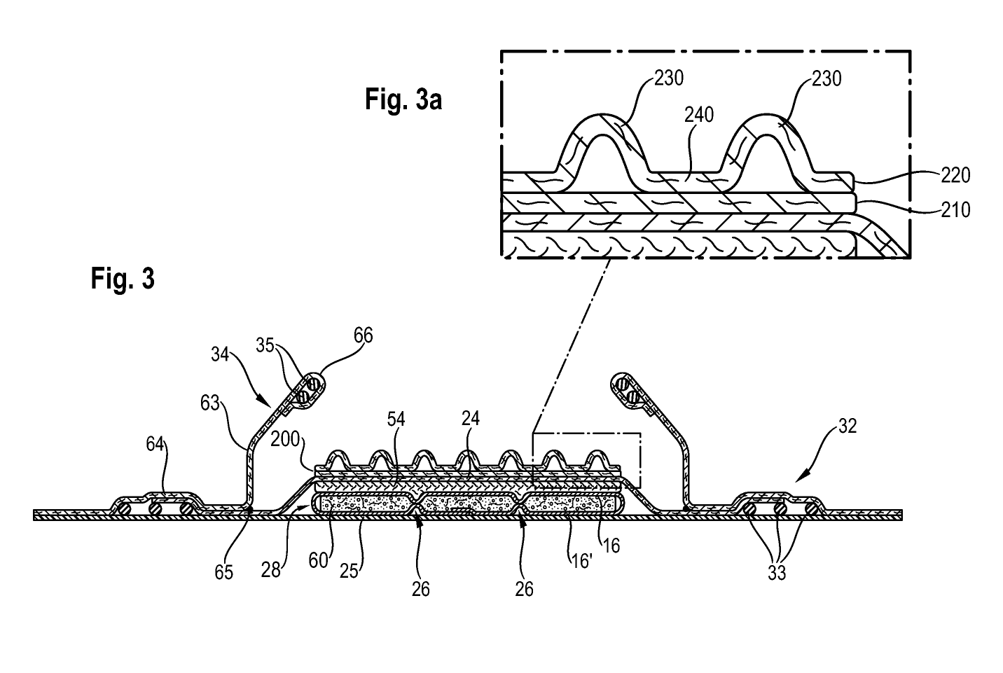

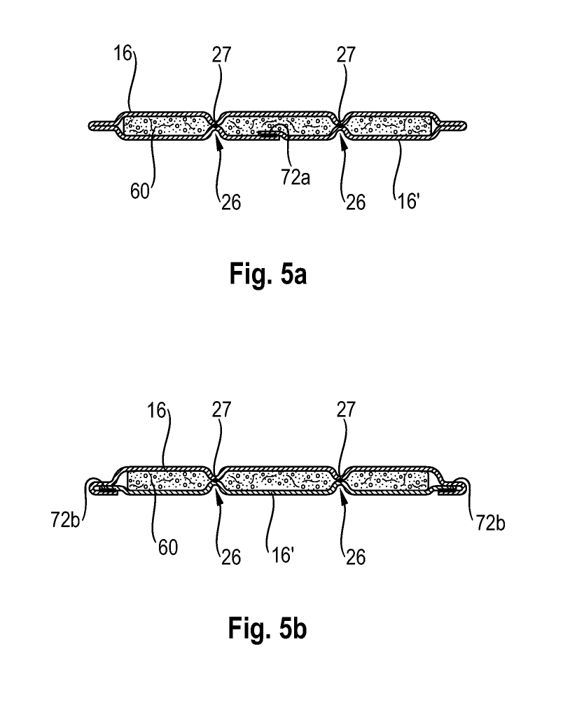

The absorbent article further comprises an absorbent core 28 between the topsheet and the backsheet. The absorbent core 28 typically comprises a mixture of fibers and superabsorbent polymer particles enclosed in a core wrap. As represented in FIG. 5a, a single substrate material may be completely wrapped around the absorbent material 60 and be bonded to itself by an adhesive core wrap bond 72a along an overlap for example on the bottom side of core wrap. The core wrap may alternatively comprise two separate nonwoven substrates forming the top side 16 and bottom side 16′ respectively of the core wrap as shown in FIG. 5b, with one substrate being wider than the other to form flaps which are wrapped around and bonded to the other substrate in a C-wrap configuration. The absorbent core may comprise as illustrated at least a pair of generally longitudinally-extending channels 26 which are substantially free of absorbent material and through which the core wrap is advantageously bonded to itself. The absorbent material 60 defines an absorbent material area 8 within the core wrap as seen from the top of the core. The absorbent material area may be advantageously shaped to define two recesses on each of the longitudinally-extending side edges 284, 286 of the core towards its crotch area similar to a dog-bone or glass-hour shape as illustrated on FIG. 3. The absorbent material area may also be rectangular with straight longitudinal side edges.

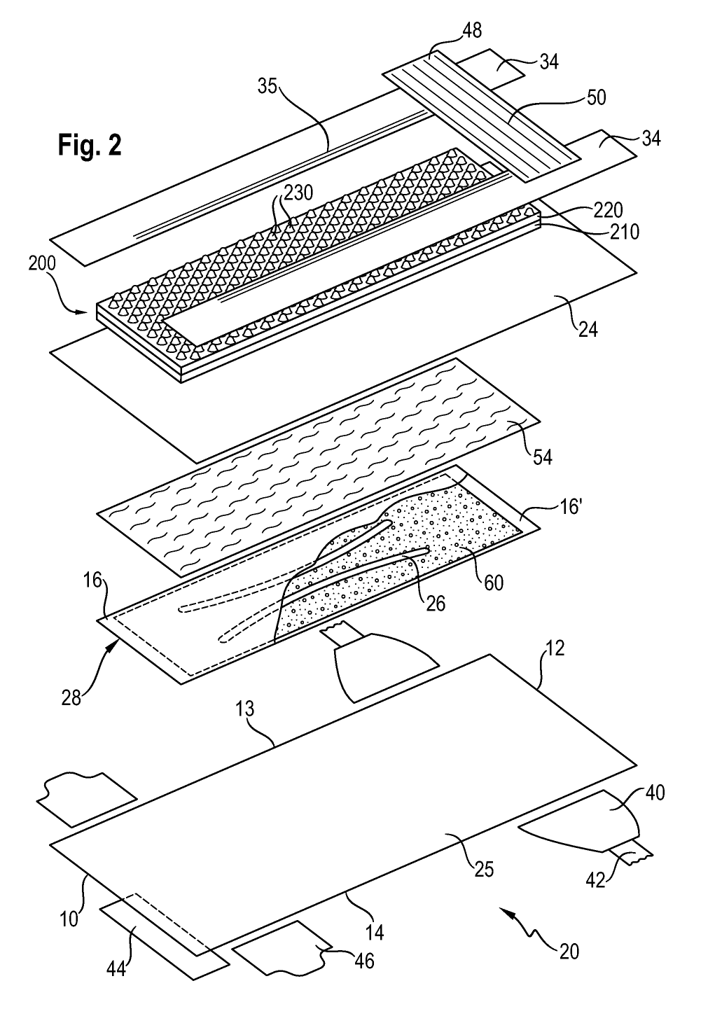

FIG. 2 and FIG. 3 show respectively an exploded view of the main components of the diapers and a cross-section of the diaper, showing a three-dimensional wearer-facing material 200, the liquid permeable topsheet 24, the liquid impermeable backsheet 25, the absorbent core 28, the acquisition layer 54 and other typical diaper components. The absorbent article may typically comprise a pair of partially upstanding barrier leg cuffs 34 and elasticized gasketing cuffs 32 substantially planar with the chassis. Both types of cuffs are typically joined to the chassis of the absorbent article typically via bonding to the topsheet and/or backsheet. The absorbent article may also comprise a wetness indicator (not shown), such as a composition comprising a hot-melt adhesive and pH-indicating agent placed on the internal side of the backsheet film or the external side of the bottom side of the core wrap. The wetness indicator may be a line generally aligned with the longitudinal axis, but it may also comprise several lines or more complex comprising discrete decorative elements such as images of toys, animals etc. Examples of wetness indicators are further disclosed for example in WO2015/095514 (Laveeta). The wetness indicator may be disposed as to appear from the garment-facing side between the pair of the core's channels. Of course the articles may further comprise any typical components known in the art such as a front elastic waistband, transverse barrier cuff(s), a lotion application, etc.

### Topsheet 24

The topsheet may be any suitable material known in the art for use as a topsheet. The topsheet should be compliant, soft-feeling, and non-irritating to the wearer's skin. Further, at least a portion of the topsheet is liquid permeable, permitting liquids to readily penetrate through its thickness. The topsheet may typically be a nonwoven or an apertured film. An example of topsheet comprises a web of spunbond polypropylene fibers. Typical diaper topsheets have a basis weight of from about 10 to about 28 gsm, in particular between from about 12 to about 18 gsm but other basis weights are possible. Suitable formed film topsheets are for example described in U.S. Pat. No. 3,929,135, U.S. Pat. No. 4,324,246, U.S. Pat. No. 4,342,314, U.S. Pat. No. 4,463,045, and U.S. Pat. No. 5,006,394. Other suitable topsheets may be made in accordance with U.S. Pat. No. 4,609,518 and U.S. Pat. No. 4,629,643.

Although not shown in the drawings, it is possible and advantageous to bond the topsheet directly or indirectly to the underlying acquisition layer. These layers may be bonded by any known bonding means, such as slot gluing, spiral gluing, fusion point bonding, or otherwise attached.

### Acquisition Layer 54

The absorbent article may comprise an acquisition layer 54 between the topsheet and the absorbent core. The acquisition layer may be the only layer between topsheet and core, but it is not excluded that there may be additional layers for example a distribution layer between the acquisition layer and the absorbent core. This acquisition layer, sometimes referred to as secondary topsheet, may for example be a through air-bonded carded web (“TABCW”) but many other alternatives material are known in the art and may be used instead. “Bonded carded web” refers to webs that are made from staple fibers that are sent through a combing or carding unit, which breaks apart and aligns the staple fibers in the machine direction to form a generally machine direction-oriented fibrous nonwoven web. This web is then drawn through a heated drum, creating bonds throughout the fabric without applying specific pressure (through air bonding process). The TABCW material provides a low density, lofty through air bonded carded web. The web may in particular have a specific weight basis level at about 15 to about 70 gsm (gram per m²). The TABCW material can for example comprise about 3 to about 10 denier staple fiber. Examples of such TABCW are disclosed in WO2000/71067 (KIM DOO-HONG et al.). TABCW are available directly from all usual suppliers of nonwoven webs for use in absorbent articles, for example Fitesa Ltd or Fiberweb Technical Nonwovens.

### Absorbent Core 28

As used herein, the term “absorbent core” refers to the component of the article which comprises an absorbent material enclosed in a core wrap and used to absorb and retain most of the liquid exudates. The absorbent core is typically the component of an absorbent article that has the most absorbent capacity of all the components of the absorbent article, and which comprises all, or at least the majority of, superabsorbent polymer (SAP). As used herein, the term “absorbent core” does not include the topsheet, the backsheet and (if present) any acquisition-distribution layer or multilayer system, which is not integral part of the absorbent core. The terms “absorbent core” and “core” are herein used interchangeably.

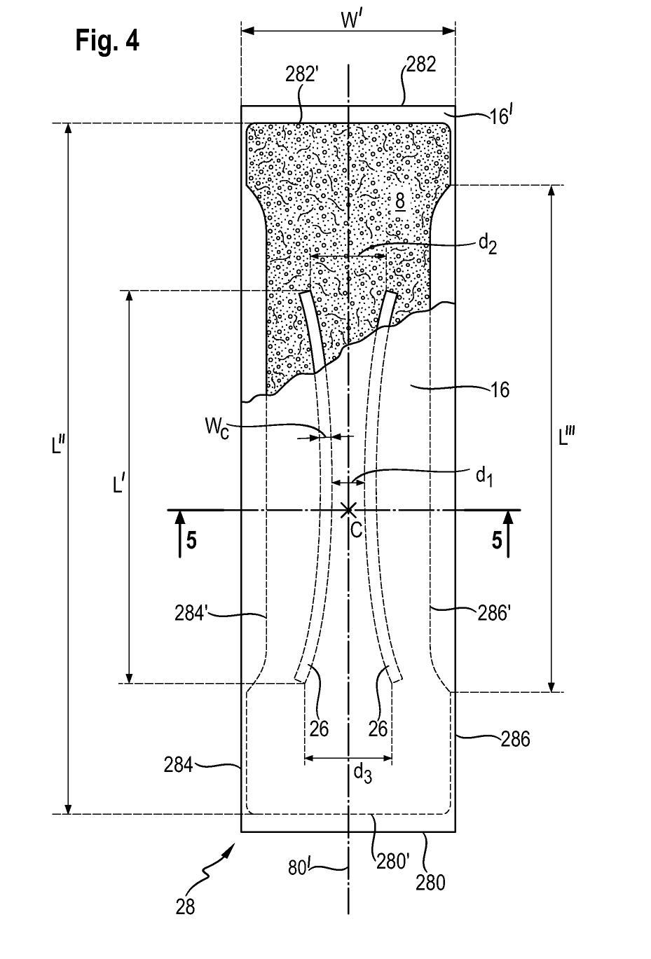

An exemplary core 28 comprising channels is represented in FIGS. 4-5 in a dry state and in isolation of the absorbent article. Absorbent cores can typically be laid flat on a surface as shown on FIG. 4, but of course they can also be laid on a non-flat surface for example a drum during their making process or stored as a continuous roll of stock material before being converted into an absorbent article. For ease of discussion, the exemplarily absorbent core of FIG. 4 is represented in a flat state and extending in a longitudinal direction parallel to the longitudinal axis 80′ of the absorbent core and a transversal direction perpendicular to the longitudinal direction. The longitudinal axis 80′ of the core may be generally parallel and contiguous to the longitudinal axis 80 of the article. Unless otherwise indicated, dimensions and areas disclosed herein apply to the core in this flat-out configuration. The same applies to the absorbent article in which the core is integrated.

The absorbent core as delimited by the core wrap 16, 16′ is typically rectangular with a front end 280, a back end 282 and two longitudinally extending side edges 284, 286. The core has a width W′ as measured in the transversal direction and a length L″ as measured in the longitudinal direction, from edge to edge including the region of the core wrap which does not enclose the absorbent material. The front end and back end may or may not be sealed. The width and length of the core may vary depending on the intended usage. For baby and infant diapers, the width W′ may for example in the range from 40 mm to 200 mm and the length L″ from 100 mm to 500 mm, as measured along the longitudinal axis 80′ of the core. In case the core is not rectangular, the maximum dimension measured along the transversal and longitudinal direction can be used to report the length and width of the core.

The core wrap comprises a top side 16 oriented towards the wearer-facing side of the article and a bottom side 16′ oriented towards the garment-facing side of the article. The core wrap may be formed of a single web wrapped around the absorbent material with one longitudinal seal 72a to attach overlapping portions of the substrate to each other, as exemplary shown on FIG. 5a. The top and bottom sides may also be formed by two separate substrates which may be the same or different material (the top layer being for example hydrophillically treated). These two substrates may be partially attached together in particular by gluing the flaps of the wider material to the other material to form two so-called C-wrap seals 72b extending longitudinally of the core as exemplary shown on FIG. 5b. This gluing may be for example provided by two slots of glue. Independent of the construction, the core wrap material may be any suitable material used in the field, typically a nonwoven web, such as a laminate comprising spunbond (“S”) or meltblown (“M”) layer. For example spunmelt polypropylene nonwovens are suitable, in particular those having a laminate web

SMS, or SMMS, or SSMMS, structure, and having a basis weight range of about 5 gsm to 15 gsm. Suitable materials are for example disclosed in U.S. Pat. No. 7,744,576, US2011/0268932A1, US 2011/0319848A1 and US2011/0250413A1. It is also not excluded that the core wrap may be partially or entirely formed by layers having an additional function such as the backsheet, the topsheet or an acquisition layer.

The absorbent material in the core may typically comprise fibers mixed with superabsorbent polymer particles. The fibers may typically comprise wood pulp (cellulose) fibers optionally mixed with synthetic fibers. The absorbent material typically comprises from 50% to 90% of superabsorbent polymers (herein abbreviated as “SAP” also referred to as absorbent gelling material) by weight of the absorbent material. The absorbent material may for example comprise at least 55% superabsorbent polymers by weight of the absorbent material, in particular from 60% to 90% superabsorbent polymers by weight of the absorbent material, in particular from 65% to 85% superabsorbent polymers by weight of the absorbent material. It is not excluded that higher amount of SAP may be present, and in some cases it may be possible that the absorbent material comprise little or no cellulose fibers (so called airfelt-free cores).

The term “superabsorbent polymer” refers herein to absorbent material, which may be cross-linked polymer, and that can typically absorb at least 10 times their weight of an aqueous 0.9% saline solution as measured using the Centrifuge Retention Capacity (CRC) test (EDANA method WSP 241.2-05E). The SAP may in particular have a CRC value of more than 20 g/g, or more than 24 g/g, or of from 20 to 50 g/g, or from 20 to 40 g/g, or from 24 to 30 g/g. The SAP may be typically in particulate forms (superabsorbent polymer particles), but it not excluded that other forms of SAP may be used such as a superabsorbent polymer foam for example. It is not excluded that higher amount of SAP may be present, and in some cases it may be possible that the absorbent material comprise little or no cellulose fibers (so called airfelt-free cores).

The absorbent material 60 defines an absorbent material deposition area 8, as seen as in FIG. 4 from above within the plane of the core. The channels 26 are encompassed within this deposition area. The absorbent material deposition area 8 may be advantageously shaped, i.e. it is not rectangular, for example it may have a sand-hour or dog-bone shape as shown in FIG. 4, but other shapes can also be used such as a “T” or “Y”. The deposition area 8 typically has a front side 280′, a back side 282′ and two longitudinally-extending sides 284′, 286′, each respectively adjacent to the corresponding sides of the core wrap. The length of the deposition area L″ is the distance between the front side and the back side of the deposition area. The deposition area preferably shows a tapering along its width in an intermediate position between the front edge and the back edge of the core as illustrated in FIG. 4. In this way, the absorbent material deposition area may have a relatively narrow width in an area of the core intended to be placed in the crotch region of the absorbent article. Having the width of the absorbent material deposition area maximum at the front edge and/or at the back edge of the absorbent material area, and minimum at a longitudinal position between the front edge and the back edge of the absorbent material area may provide better wearing comfort for the wearer. The area of minimum width intermediate the front side 280′ and the back side 282′ of the absorbent material deposition area, including any transition areas, may for example have a length (L′″) which ranges from 10% to 80% of the length of the deposition area (L″) as measured in the longitudinal direction.

### Channels 26

The absorbent cores of the invention comprise at least one, and advantageously at least one pair, of longitudinally-extending channels 26. The channels are defined by areas within the absorbent material deposition area 8 that are substantially free of absorbent material. The absorbent core may in particular comprise a pair of channels symmetrically placed relative to the longitudinal axis, but it is not excluded that only one channel may be present, or more than a pair of channels. By “substantially free” it is meant that in the channel areas the basis weight of the absorbent material is at least less than 25%, in particular at least less than 20% or less than 10%, of the average basis weight of the absorbent material in the rest of the absorbent material deposition area. In particular there can be no absorbent material in the channels. Minimal amount such as involuntary contaminations with absorbent material that may occur during the making process are not considered as absorbent material. The channels 26 are advantageously surrounded by the absorbent material, when seen in the plane of the core as seen on FIG. 4, which means that the channels do not extend to any of the edge of the deposition area 8 of the absorbent material. Typically, the smallest distance between a channel 26 and the closest edge of the absorbent material deposition area 8 is at least 5 mm.

The top side 16 of the core wrap may be attached to the bottom side 16′ of the core wrap through the channels by one or a plurality of channel bonds 27. Various bonding means can be used such as ultrasonic, heat (fusion), mechanical or adhesive bonding. Ultrasonic bonding may be particular useful in terms of reduced raw material usage and strength of the bond. When the absorbent material swells upon absorbing a liquid, the channel bonds 27 within the channels remains at least initially attached in the channels. The absorbent material swells in the rest of the core, so that the core wrap forms more marked three-dimensional channels along each channel 26 where the channel bonds 27 are present. These channels can distribute an insulting fluid along their length to a wider area of the core and thus provide a quicker fluid acquisition speed and a better utilization of the absorbent capacity of the core. The three-dimensional channels can also provide a deformation of an overlying layer such as a fibrous acquisition layer 54 and provide corresponding ditches in the overlying layer (see WO2014/200794A1). The channel bond 27 may be a continuous bond extending along each of the channels 26, but the channel bond in each channel is typically discontinuous (intermittent) such as series of point bonds.

The following are examples of shape and size of channels, but are not limiting the scope of the invention. In general, the channel bonds may have the same outline but be slightly smaller than the channels due to the tolerance required in some manufacturing process. The channels may be present within the crotch region of the article, as defined as being the longitudinally middle third of the article. The absorbent core may also comprise more than two channels, for example at least 3, or at least 4 or at least 5 or at least 6.

The channels extend generally longitudinally, which means that each channel area extends at least as much in the longitudinal direction as in the transverse direction, and typically at least twice as much in the longitudinal direction than in the transverse direction (as measured after projection on the respective axis). The absorbent core, as illustrated in FIG. 4, typically also have a longitudinal axis 80′ which is contiguous with the longitudinal axis 80 of the article. The channels 26 may have a length L′ projected on the longitudinal axis 80′ of the core that is at least 10% of the length L of the absorbent article, in particular from 20% to 80%. The channels 26 may be for example have a length L′ of at least 2 cm as measured on the longitudinal axis, or at least 4 cm, 6 cm, 8 cm, or 10 cm, and for example up to 40 cm, or 30 cm. Shorter channels may also be present in the core, for example in the back region or the front region of the core, as seen for example in the Figures of WO2012/170778.

Each channel may have a width Wc along at least part of its length which is at least 2 mm, or at least 3 mm or at least 4 mm, up to for example 20 mm, or 16 mm or 12 mm. The width Wc of each channel 26 may be constant through substantially its whole length or may vary along its length. The channels may be straight and longitudinally oriented parallel to the longitudinal axis. The channels may also be inwardly curved (concave) towards the longitudinal axis 80/80′, as for example represented in FIG. 4. Thus the channels may have a closest distance d1 at their closest, and a farthest distance d2, d3. The farthest distance is typically the distance d2 between the both back extremities of the channels, or the distance d3 between the two front extremities of the channels, whichever is the longest. In the example represented, the channels extend so that the distance d2 is about equal to d3. The channels may be sufficiently curved so that the closest distance d1 is at least less than 80% of the farthest distance d2, d3, preferably wherein the closest distance ranges from 10% to 70% of the farthest distance. This can be summarized by the equation:

d1<0.80 max(d2, d3)

in particular:

0.10 max(d2, d3)≦d1≦0.70max(d2, d3)

wherein max (d2, d3) means the largest of d2 or d3 (d2 and d3 can also be about equal).

The radius of curvature may be constant for a channel, or it may vary along its length. The channels may be comprised of a series of generally straight segments approximating a curve (for example three linked segments, the middle one being straight and longitudinally oriented and the two remaining one tilted relative to the longitudinal axis) instead of being a smooth curve as represented. The channels 26 typically do not coincide with the longitudinal axis 80/80′ of the article/core. The smallest spacing distance d1 between the channels forming a pair may be for example at least 5 mm, or at least 10 mm, or at least 16 mm.

As the absorbent core absorbs liquid, the depressions formed by these channels will become deeper and more apparent to the eye and the touch from the exterior of the article as the backsheet and topsheet are pushed outwardly by the expending absorbent material. If the channel bond 27 is sufficiently strong and the level of SAP is not too high, it is possible that the channel bonds remain permanent until complete saturation of the absorbent material. On the other hand, the channel bonds may in some cases also restrict the swelling of the absorbent material when the core is substantially loaded. The channel bond 27 may also be designed to open in a controlled manner when exposed to a large amount of fluid. The bonds may thus remain substantially intact at least during a first phase as the absorbent material absorbs a moderate quantity of fluid. In a second phase the channel bonds 27 in the channels can start opening to provide more space for the absorbent material to swell while keeping most of the benefits of the channels such as increased flexibility of the core in transversal direction and fluid management. In a third phase, corresponding to a very high saturation of the absorbent core, a more substantial part of the channel bonds can open to provide even more space for the swelling absorbent material to expand. The strength of the channel bonds 27 within the channels can be controlled for example by varying the number and intensity of the point bonds attaching the two sides of the core wrap and/or the distribution of the superabsorbent material, as more absorbent material will usually causes more swelling and will put more pressure on the bond. The extensibility of the material of the core wrap may also play a role.

### Waistband 48

As illustrated in FIG. 1, the article 20 also has an elastic back waistband 48 extending transversally adjacent the back waist edge 12 of the article. Such waistbands (also called elastic waist features) may typically comprise a nonwoven substrate and a plurality of elastic strands 50 transversally orientated. Typical waistbands comprise extruded strand elastomer between two layers of spunbond nonwoven; e.g. using PP fibers or bi-component core/sheath PE/PP or PE/PET fibers. Other types of substrates may be used if desired. Spandex (=Elastane or LYCRA®) strands may also be used as elastics between the nonwovens. Other executions of applied waistband consist of elastics stretched in the process and applied transversely to the length of the articles directly sandwiched in between some wearer-facing and some garment-facing material.

The waistbands in cooperation with the other features of the invention results in absorbent articles having increased comfort, fit, and improved leakage performance for the wearer. As indicated previously, it was found that absorbent core with longitudinally-extending channels may provide an increased rigidity in the longitudinal direction when the absorbent material has swollen and presses against the walls of the core wrap defining the channels. This may create further gaps towards the back edge of the article. The present invention delivers an absorbent article having improved gap closure in at least the back waist region of the absorbent article using the combination of the stretchable waistband and the stretchable back ears. The article may comprise, in addition to the back waistband 48, a front elasticized waistband (not represented). In the following, the description referring to the back waistband may also refer independently to the front waistband, unless specifically indicated otherwise. “Stretchable”, “elastic”, “elastically extensible”, and “elasticized” refer herein to the property of a material and/or an element of a diaper or other disposable absorbent article whereby the material and/or the element can be elongated to at least 150% of its original un-stretched length without rupture or catastrophic failure upon the application of tensioning force and will substantially return to its original length or near its original length after the tension is released.

The waistband 48 typically comprises a laminate of a nonwoven and several elastic strands 50 that are combined with the chassis under some tension. Elastic strands are the most cost effective way to get stretch that exhibits little relax or set over time. Nonwovens are preferred for the exterior of the waist band material because it is breathable and softer than film alternatives, but films may also be used as waistband material. The waistband laminate may further comprise any number of strands are as desired, for example from 2 elastic strands to 40 elastic strands, for example from 4 elastic strands to 26 elastic strands. It is also known that when strands of elastic are combined under strain with other often non-extensible materials and then allowed to relax, they will create a laminate that has gathers of a certain frequency and a resulting basis weight that is higher than the starting materials laid flat. Non-limiting examples of back and front waistbands can be found in WO2012/177400 and WO2012/177401 (Lawson), and U.S. Pat. No. 4,515,595, U.S. Pat. No. 4,710,189, U.S. Pat. No. 5,221,274 and U.S. Pat. No. 6,336,922 (VanGompel et al.).

The nonwoven material and the elastic strand(s) may be combined under a first strain (Installed Strand Elongation) and the waistband is attached to the article under a second strain (Applied Waistband Strain). The nonwoven material and the elastic strand(s) may be combined with adhesive, mechanical bonds, or any other forms of attachment known in the art.

The Installed Strand Elongation may be greater than about 50%, greater than about 75%, greater than about 100%, greater than about 150%, greater than about 200%, greater than about 225%, greater than about 250%, greater than about 300%, greater than about 350%, greater than about 375%. The installed elongation is the strain at which the elastic is under relative to the second material that it is combined with (e.g. low basis weight nonwoven). For example, if the elastic is stretched from 100 mm to 250 mm when it is combined with the nonwoven, it would be said to be 150% installed elongation or ((250 mm/100 mm)−1)×100%. This laminate can then be allowed to relax and will return to about the original 100 mm, but with 250 mm of nonwoven. There can be more than one installed elongation within one waistband laminate if the elastics are strained to a different degree. For example, strand (1) is stretched from 100 mm to 250 mm when combined with the nonwoven or has 150% installed elongation while strand (2) is stretched from 90 mm to 250 mm when combined with the NW or has an installed elongation of about 178%.

The waistband may be applied to the disposable absorbent article at an Applied Waistband Strain of greater than about 30%, greater than about 50%, greater than about 70% as compared to the relaxed length. The waistband may be applied to the disposable absorbent article at an Applied Waistband Strain of less than about 150%, less than about 125%, less than about 100%, less than about 75% as compared to the relaxed length. The Applied Waistband Strain is the strain that the waistband laminate is under when combined with the absorbent article. For example if 100 mm of laminate is stretched to 170 mm when applied it would be considered to be 70% applied waistband strain or ((170 mm-100 mm)/100 mm×100%). If a front waistband is present, it may be applied advantageously to the chassis at the same Applied Waistband Strain as the backsheet, or at a different strain.

The waistband may have a length in the direction parallel to the longitudinal axis of the article of greater than about 12 mm, greater than about 15 mm, greater than about 20 mm. The waistband may have a length in the direction parallel to the longitudinal axis of the article f less than about 50 mm, less than about 45 mm, less than about 40 mm. The waistband in a relaxed product may have a length in the direction parallel to the transversal axis of the article of greater than about 50 mm, greater than about 75 mm, greater than about 100 mm. The length in the direction parallel to the transversal axis of the article of the waistband in a relaxed product may be less than about 300 mm, less than about 250 mm, less than about 200 mm. The waistband is typically disposed on the wearer-facing surface of the article, but it is not excluded that the waistband may be on the garment-facing surface of the article. The waistband may be also sandwiched in between the layers of the absorbent article.

The waistband is generally placed adjacent the corresponding waist edge of the article. The distance between the waistband and the edge of the article may be in particular less than 40 mm, in particular the distance between the (back) waistband and the (back) edge of the article may be from 0 mm to 30 mm. The waistband may be attached to the article with adhesive, mechanical bonds, or any other forms of attachment known in the art.

### Backsheet 25

The backsheet may be any backsheet known in the art for absorbent articles. The backsheet may be positioned directly adjacent the garment-facing surface of the absorbent core. The backsheet prevents, or at least inhibits, the exudates absorbed and contained therein from soiling articles such as bedsheets and undergarments. The backsheet is typically impermeable, or at least substantially impermeable, to liquids (e.g., urine). The backsheet may, for example, be or comprise a thin plastic film such as a thermoplastic film having a thickness of about 0.012 mm to about 0.051 mm. Example backsheet films include those manufactured by Tredegar Corporation, based in Richmond, Va., and sold under the trade name CPC2 film. A covering low basis weight nonwoven may be attached to the external surface of the film to provide for a softer touch.

Suitable backsheet materials include breathable materials which permit vapors to escape from the absorbent article while still preventing, or at least inhibiting, exudates from passing through the backsheet. Example breathable materials may include materials such as woven webs, nonwoven webs, composite materials such as film-coated nonwoven webs, microporous films such as manufactured by Mitsui Toatsu Co., of Japan under the designation ESPOIR NO and by Tredegar Corporation of Richmond, Va., and sold under the designation EXAIRE, and monolithic films such as manufactured by Clopay Corporation, Cincinnati, Ohio under the name HYTREL blend P18-3097.

The film may include at least about 20 weight percent filler particles, for example filler particles that include calcium carbonate, so that wherein the film has been stretched in the machine direction, e.g. to at least about 150 percent, fractures are formed where said filler particles are located. The films may be biaxially stretched at least about 150 percent in the machine direction and a transverse direction to cause fractures to form where said filler particles are located. Breathable films may generally have Water Vapor Transmission Rates (WVTR) in excess of 300 grams per square meter per 24 hours. The WVTR may be measured by the Desiccant Method as indicated in ASTM E96/E96M-14.

U.S. Pat. No. 6,075,179 for example discloses a suitable multilayer film comprising: a core layer made from an extrudable thermoplastic polymer, the core layer having a first exterior surface and a second exterior surface, a first skin layer attached to the first exterior surface of said core layer to form the multilayer film, the multilayer film defining an overall thickness. The first skin layer defines a first skin thickness, and comprising less than about ten percent of said overall thickness. The overall thickness is not exceeding about 30 micrometers and the multilayer film is a liquid barrier and has a WVTR of at least 300 g/m²/24 hours.

The backsheet may further typically comprise a nonwoven on its most external side to improve softness. Exemplary laminates comprising a breathable film and a nonwoven layer are for example disclosed in WO2014/022,362A1, WO2014/022,652A1 and U.S. Pat. No. 5,837,352. The nonwoven web may in particular comprise a spunbond nonwoven web and/or a laminate of a spunbond nonwoven web and a meltblown nonwoven web. The laminate may also have a water vapor transmission rate of at least 300 g/m²/24 hours. U.S. Pat. No. 5,843,056 for example discloses substantially liquid impermeable, vapor permeable composite backsheet that comprises:

in a first embodiment, a) a substantially liquid impermeable, vapor permeable film which defines a basis weight of less than about 20.0 grams per square meter; and b) a nonwoven facing layer which is attached to a garment facing surface of said film and which defines a strength in a machine direction of at least about 3000 grams at an elongation of 30 percent;

in a second embodiment, a polymeric film which defines a basis weight of less than about 20.0 grams per square meter and a nonwoven facing layer attached to a garment facing surface of said film, wherein said composite backsheet defines a water vapor transmission rate of at least about 500 g/m²/24 hour and a hydrohead value of at least about 50 centimeters as determined according to Method 5514-Federal Test Methods Standard No. 191A; and

in a third embodiment, a) a polyethylene film which includes at least about 20 weight percent filler particles and which defines a basis weight of less than about 20.0 grams per square meter; and b) a meltblown facing layer which is attached in facing relation to a garment facing surface of said film and which defines basis weight of less than about 30.0 grams per square meter and a hydrohead value of at least about 50 centimeters; wherein a ratio of a strength of said composite backsheet to a strength of said film in a machine direction at an elongation of 30 percent is at least about 2:1.

### Back Ears 40, Fastening Tabs 42 and Landing Zone 44

The absorbent article comprises stretchable back ears 40 each comprising attached thereto at least one fastening tab 42. The fastening tabs 42 can be releasably engaged with the landing zone 44 disposed on the garment-facing side of the article adjacent the front edge of the article. The back ears, fastening tabs and the landing zone may be any standard components known in the art.

The ears may be an integral part of the chassis, such as formed from the topsheet 24 and/or backsheet 26 as side panel but more typically, as represented on FIG. 1, the ears may be separate elements attached by gluing, heat embossing, and/or pressure bonding to the rest of the article. The back ears 40 are stretchable to facilitate the attachment of the fastening tabs 42 to the landing zone 44 and maintain the taped diapers in place around the wearer's waist. The stretchable back ears 40 also provide a more comfortable and contouring fit by initially conformably fitting the absorbent article to the wearer and sustaining this fit throughout the time of wear well past when absorbent article has been loaded with exudates since the elasticized ears allow the sides of the absorbent article to expand and contract. The back ears may in particular be a stretchable laminate comprising an elastomeric film sandwiched between two nonwoven layers. The nonwoven layer may for example be a spunbond nonwoven (e.g. PP or PE/PP bico fibers) and the film a SBC/POE mix. The basis weight of the laminate may typically range from 50 gsm to 200 gsm, for example 110 gsm or 150 gsm. The nonwoven may typically have a nonwoven basis weight ranging from 20-30 gsm and the film's basis weight may typically range from 35-45 gsm.

The fastening tabs are typically a plastic material attached to the back ears and may for example comprise “Velcro-type” hooks that can releasably engage with loops disposed on the landing zone 44. Many different systems have been suggested in the art. Some exemplary surface fastening systems are disclosed in U.S. Pat. No. 3,848,594 entitled “Tape Fastening System for Disposable Diaper” issued to Buell; U.S. Pat. No. 4,662,875 issued to Hirotsu et al.; U.S. Pat. No. 4,846,815 entitled “Disposable Diaper Having An Improved Fastening Device” issued to Scripps on Jul. 11, 1989; U.S. Pat. No. 4,894,060 entitled “Disposable Diaper With Improved Hook Fastener Portion” issued to Nestegard; U.S. Pat. No. 4,946,527 entitled “Pressure-Sensitive Adhesive Fastener And Method of Making Same” issued to Battrell on Aug. 7, 1990; U.S. Pat. No. 5,151,092 and U.S. Pat. No. 5,221,274. An exemplary interlocking fastening system is disclosed in U.S. Pat. No. 6,432,098 entitled “Absorbent Article Fastening Device” in the names of Kline et al.

The landing zone 44 may for example be a pattern-unbonded nonwoven fabric as disclosed in U.S. Pat. No. 5,858,515 and comprising a first nonwoven web having a fibrous structure of individual fibers or filaments. The nonwoven web may have a bulk of at least about 10 mils and a basis weight of at least about 20 grams per square meter. The nonwoven web may have on a surface thereof a pattern of continuous bonded areas defining a plurality of discrete unbonded areas formed by application of heat and pressure; with a percent bond area of from about 25 percent to about 50 percent; the individual fibers or filaments within the discrete unbonded areas having at least a portion thereof extending into and bonded within said continuous bonded areas.

### Cuffs 32, 34

The absorbent articles may typically further comprise components that improve the fit of the article around the legs of the wearer, in particular a pair of barrier leg cuffs 34 and gasketing cuffs 32. The barrier leg cuffs 34 may each be formed by a piece of material, typically a nonwoven, that can be partially raised away and thus stand up from the plane defined by the topsheet, as shown for example in FIG. 2. The material of the barrier leg cuffs may thus comprise a first portion 64 flush with the topsheet and limited inwardly by a proximal edge 65. This first portion 64 may be attached to the topsheet and/or backsheet with an intermittent or continuous fusion bond and/or a glue bond. The barrier leg cuffs 34 further comprise a free-standing portion 63 limited by a distal edge 66, which in use fits at the junction of the thighs with the torso of the wearer, at least in the crotch region of the article. The barrier leg cuffs can provide improved containment of liquids and other body exudates approximately at the junction of the torso and legs of the wearer. Typically, the barrier leg cuffs are formed from a separate material joined to the rest of the article, in particular to the topsheet, but it is not excluded that the barrier leg cuffs can be integral with (i.e. formed from) the topsheet or the backsheet, or any other layer, for example the bottom layer of the core wrap. Typically the material of the barrier leg cuffs may extend through the whole length of the article but is further bonded to the topsheet towards the front edge and back edge of the article so that in these sections the barrier leg cuff material remains flush with the topsheet (tack bonds not shown in FIG. 1 for readability). Each barrier leg cuff 34 typically comprises one, two or more elastic strings 35 close to the free standing terminal edge 66.

In addition to the barrier leg cuffs 34, the article may typically comprise gasketing cuffs 32, which may be present as part of the chassis of the absorbent article. The gasketing cuffs may be at least partially enclosed between the topsheet and the backsheet, or the barrier leg cuffs and the backsheet. The gasketing cuffs may be placed transversally outward relative to the proximal edge 65 of the barrier leg cuffs 34. The gasketing cuffs 32 can provide a better seal around the thighs of the wearer. Usually each gasketing cuff 32 will comprise one or more elastic string or elastic element(s) 33 embedded within the chassis of the diaper, for example between the topsheet and backsheet in the area of the leg openings. These elastic elements 33 may, independently or in combination with the elastics 35 of the barrier leg cuffs, help shaping the absorbent article into a basin shape when put in place on and being worn by the wearer.

Various cuff constructions have been disclosed for in the art and may be used in the present invention. U.S. Pat. No. 3,860,003 describes a disposable diaper which provides a contractible leg opening having a side flap and one or more elastic members to provide gasketing cuffs. U.S. Pat. No. 4,808,178 and U.S. Pat. No. 4,909,803 (Aziz) describe disposable diapers having “stand-up” elasticized flaps (barrier leg cuffs) which improve the containment of the leg regions. U.S. Pat. No. 4,695,278 (Lawson) and U.S. Pat. No. 4,795,454 (Dragoo) describe disposable diapers having dual cuffs, including gasketing cuffs and barrier leg cuffs. More recently, WO2005/105010 (Ashton) discloses a dual cuff system made of a continuous cuff material. All or a portion of the barrier leg and/or gasketing cuffs may be treated with a lotion.

### Three-Dimensional Wearer-Facing Material 200

The article may comprise on its wearer-facing side a three-dimensional material 200, in particular but not limited to a dual layered three-dimensional material as described in further details below. This three-dimensional material may by itself form a topsheet, or may be attached to an underlying topsheet 24 having a larger area.

As illustrated in FIGS. 3-3a, the articles of the invention may comprise a dual-layer wearer-facing material 200. This three-dimensional material is placed above the topsheet 24 and comes directly in contact with the skin of the wearer. The three-dimensional material may be attached to the topsheet, typically using an adhesive for example an adhesive having a spiral application pattern. The wearer-facing material may be typically shorter in the transversal and in longitudinal direction than the topsheet, but it is not excluded that it may be as long and/or as wide as the topsheet. The wearer-facing material comprises a fibrous support layer 210 and a fibrous projection layer 220. The projection layer comprises a plurality of fibrous projections 230 extending from the outer surface of the projection layer in a direction away from the support layer and formed from a first plurality of fibers in the projection layer. The projection layer will be at least partially in contact with the wearer's skin when the article is worn. The projections 230 are at least partially hollow, but can also be partially filled with fibers from the projection layer and/or the support layer.

The support layer 210 has an inner surface and an outer surface. As used herein, “inner” means oriented towards the interior of the article and “outer” means the opposite side, i.e. facing towards the exterior of the article. The projection layer 220 also has an inner surface and an opposed outer surface. At the interface between the support and the projection layer, fibers of the projection layer and the support layer are advantageously entangled with each other so that the support and the projection layer form a unitary material that can be easily handled and attached to the topsheet. This entanglement of a second plurality of fibers of the projection layer and the support layer in the land areas 240 may be a direct result of the fluid-entanglement process used to form the projections on a forming surface comprising holes. Examples of such dual layered three-dimensional material and processes to obtain them have been disclosed for example in US2014/0121623A1 (see also US2014/0121621A1, US2014/0121624A1, US2014/0121625A1, which are all incorporated herein by reference for the purpose of describing the three-dimensional wearer-facing material). The wearer-facing material 200 can be a fluid entangled laminate web.

The projections 230 typically have closed ends which are devoid of apertures, but it is not excluded that one or more apertures may be present in each of the projections. The projections can be generally rounded when viewed from above, with somewhat domed or curved tops or closed ends, such as seen when viewed in a cross-section such as shown in FIG. 3A. The actual shape of the projections can be varied depending on the shape of the forming surface into which the fibers from the projection layer are forced. Both the width and height of the projections can be varied as can be the spacing and pattern of the projections. It is also not excluded that projections of different shapes, sizes and spacing of the projections can be utilized in the same projection layer, however the projections 230 of one projection layer may be advantageously substantially similar to another. The projections can for example have a height of greater than about 0.5 mm, in particular between 1 mm and 5 mm, as measured with the Projection Height Measurement Method indicated below. The average peak-to-peak projections distance may be in the mm range, in particular from about 3 mm to 10 mm. The average number of projections per unit of surface of the three-dimensional material may be from 1 to 20 projections per square centimeter, in particular from 2 to 10 projections, such as form 2.5 to 7 projections.

The projections 230 are separated from each other and surrounded by land areas 240 which can be part of the outer surface of the projection layer, although the thickness of the land areas is typically comprised of both the projection layer and the support layer. The land areas can be relatively flat and planar, as shown in FIG. 3. It is also possible that at least some of the land areas may be provided with depressions which can extend all or part way into the projection layer and/or the support layer. The projections may be typically regularly disposed on the whole of the projection layer, for example the projections can be formed by a series of rows oriented longitudinally and/or transversally, each row being shifted from the next row by half a period, as illustrated on FIG. 1. This and further embodiments can be found in US2014/0121621A1, in particular FIG. 9 and the accompanying description, incorporated herein by reference.

The support layer 210 is typically a fibrous nonwoven web that can support the projection layer containing the projections. It can be made from a number of structures, provided the support layer is capable of supporting the projection layer. The primary functions of the support layer can be to protect the projection layer during the formation of the projections, to be able to bond to or be entangled with the projection layer and to aid in further processing of the projection layer and the resultant wearer-facing material. The support layer may be formed for example from a spunbond polypropylene web having a basis weight of from 10 gsm to 40 gsm. The spunbond web may be point bonded.

The projection layer 220 can be made from a plurality of randomly deposited fibers which may be staple length fibers such as are used, for example, in carded webs, air-laid webs, coform webs, etc., or they may be more continuous fibers such as are found in, for example, meltblown or spunbond webs. The fibers in the projection layer can have less fiber-to-fiber bonding and/or fiber entanglement and thus less integrity as compared to the integrity of the support layer. The projection layer may be for example a carded web made of polyester staple fibers. The web forming the projection layer may have a basis weight of from 20 gsm to 50 gsm. This and other examples of suitable materials are further described in the references previously indicated.

In general, a fluid entangling process can be employed to form the wearer-facing material. The most common technology used in this regard can be referred to as spunlace or hydroentangling technology which can use pressurized water as the fluid for entanglement. The apparatus can include a first transport belt, a transport belt drive roller, a projection forming surface, a fluid entangling device, an optional overfeed roller, and a fluid removal system such as a vacuum or other conventional sucking device. A detailed description of various processes and apparatuses for making the dual-layer wearer-facing material is disclosed in US2014/0121621A1 with reference to FIGS. 12-17 of this document.

The projection forming surface can be in the form of a texturizing drum having a forming surface containing a pattern of forming holes that can correspond to the shape and pattern of the desired projections in the projection layer and the forming holes can be separated by land areas. The forming holes can be of any shape and any pattern, but in particular the shapes of the holes can be round. A plurality of pressurized projection fluid streams of entangling fluid (such as water) can be directed into the laminate web comprising the support layer and the projection layer to cause a first plurality of the fibers of the projection layer to be directed into the forming holes to form the plurality of projections which extend outwardly from the outer surface of the projection layer thereby forming the fluid entangled dual-layered wearer-facing material. The laminate web can be pre-entangled before the step of forming the projections.

### Other Components

The absorbent articles of the invention can further comprise any other typical components known for the intended purpose of the article. FIG. 1 and FIG. 2 show other typical taped diaper components not further discussed herein such as front ears 46 facilitating the placement of the diaper on the wearer. The absorbent article may also comprise other known diaper components, which are not represented in the Figures, such as a front elastic waistband, transverse barrier elements across the topsheet, a wetness indicator between the core and the backsheet that changes appearance when contacted with urine, a lotion application on the topsheet, etc. These components are well-known in the art and will not be further discussed herein. Reference is made to WO2014/093310 where several examples of these components are disclosed in more details.

### Method of Making the Article—Relations Between the Layers

The absorbent articles of the invention may be made by any conventional methods known in the art. In particular the articles may be hand-made or industrially produced at high speed. The absorbent core may be made using a standard air laying drums and process adapted to provide channels and bonding through these channels. US2007/250026A1 discloses such an air-laying drum providing a plurality of holes in an unitary absorbent core. The forming drums comprise a plurality of drums forming the holes through which the core wrap can be bonded to itself. The apparatus of this document can be easily modified by changing the plurality of the nubs to at least a pair of curved and longitudinally extending strips to provide the channels of the invention. A method for making absorbent cores with channels in an airfelt-free core process is for example disclosed in WO2012/170798A1.

More generally, adjacent layers within the article will be joined together using conventional bonding method such as adhesive coating via slot coating, spiral gluing, or spraying on the whole or part of the surface of the layer, or thermo-bonding, or pressure bonding or combinations thereof. Most of the bonding between components is for clarity and readability not represented in the Figure. This bonding is exemplarily represented for the channel bond 27 between the core wrap layers within the channels 26 or the C-wrap bond(s) 72 of the core wrap. Other glues or attachments are not represented for clarity and readability but typical bonding between the layers of the article should be considered to be present unless specifically excluded. Adhesives may be typically used to improve the adhesion of the different layers, for example between the backsheet and the core wrap. The glues used may be any standard hotmelt glue as known in the art. For example, the backsheet and the core wrap may be glued using a core-to-backsheet gluing pattern as disclosed in WO2012/170341A1 (Hippe), or a full coverage pattern using several spiral glue applicators. If for example the backsheet is attached by gluing or otherwise to the areas of the core wrap corresponding to the folding guides (not shown), the folding guides may become more visible to the user from the garment-facing side of the article. Any typical hotmelt adhesives may be used. It is also possible to use a printed adhesive layer, for example between the topsheet and absorbent core or liquid management layer, which may be optionally visible through the topsheet, as exemplary disclosed in WO2014/078247.

The articles may be folded and packaged as is known in the art. In particular the articles may be packaged under compression to reduce the space occupied by the package during transport and storage. The package may be for example a plastic bag or a cardboard box. Diapers may typically bi-folded along the transversal axis and the ears folded inwardly before being packaged.

### Packages

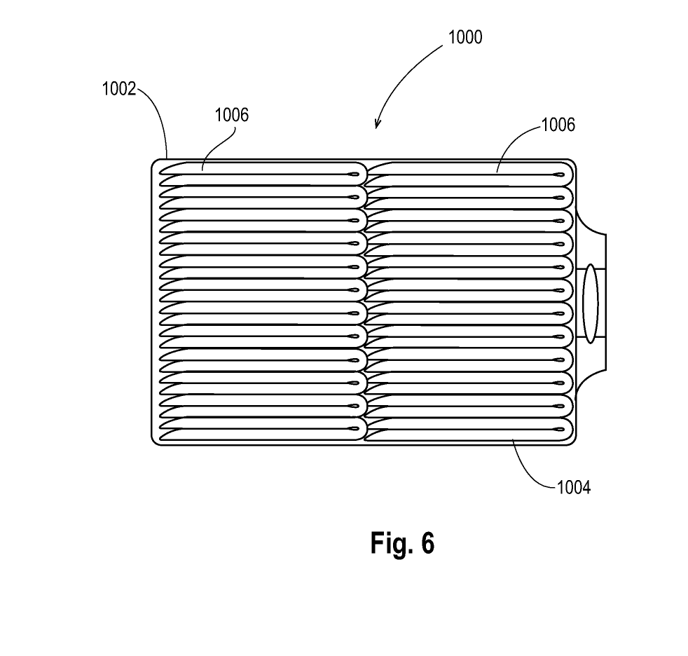

The articles may be folded and packaged as is known in the art. The package may be for example a plastic bag or a cardboard box. Diapers may typically bi-folded along the transversal axis and the ears folded inwardly before being packaged. The absorbent articles may be packed under compression so as to reduce the size of the packages, while still providing an adequate amount of absorbent articles per package. By packaging the absorbent articles under compression, caregivers can easily handle and store the packages, while also providing distribution savings to manufacturers owing to the size of the packages. FIG. 6 illustrates an example package 1000 comprising a plurality of absorbent articles 1004. The package 1000 defines an interior space 1002 in which the plurality of absorbent articles 1004 are situated. The plurality of absorbent articles 1004 are arranged in one or more stacks 1006.

The three-dimensional material 200 if present on the article may be particularly resilient to compression so that the articles may be compressed to a certain extent in the package. It is believed that the plurality of relatively closely spaced, relatively small, and relatively pillowy three-dimensional projections may act as springs to resist compression and recover once a compressive force is removed, especially in the areas in the vicinity of the channels. Compression recovery is important in nonwoven or other component layers of absorbent articles, because such articles are typically packaged and folded in compressed conditions. Manufacturers of personal care products desire to retain most, if not all of the as-made caliper for aesthetic and performance purposes. Furthermore, it is believed the channels being substantially material-free may contribute to an unexpected, beneficial improvement in compression recovery as they provide spacing for at some of the three-dimensional projections to nest in during storage and transport in the compressed package state.

The articles of the inventions may be packaged compressed at an In-Bag Compression Rate of at least 10%, in particular of from 10% to 50%, in particular from 20% to 40%. The “In-Bag Compression Rate” as used herein is one minus the height of a stack of 10 folded articles measured while under compression within a bag (“In-Bag Stack Height”) divided by the height of a stack of 10 folded articles of the same type before compression, multiplied by 100; i.e. (1-In-Bag Stack Height/stack height before compression)*100, reported as a percentage. Of course, the stack in the bag does not need to have exactly 10 articles, rather the value measured for the height of stack of article in the package is divided by the number of articles in the stack and then multiplied by 10. The method used to measure the In-Bag Stack Height is described in further details in the Test Procedures. The articles before compression may be typically sampled from the production line between the folding unit and the stack packing unit. The stack height before compression is measured by taking 10 articles before compression and packing, and measuring their stack height as indicated for the IBSH.

Packages of the absorbent articles of the present disclosure may in particular have an In-Bag Stack Height of less than 110 mm, or less than 105 mm, or less than 100 mm, or less than 95 mm, or less than 90 mm according to the In-Bag Stack Height Test described below. The In-Bag Stack Height may be at least 70 mm, at least 72 mm, at least 74 mm, at least 80 mm, according to the In-Back Stack Height Test described herein, specifically reciting all 0.1 mm increments within the specified ranges and all ranges formed therein or thereby, such as from from 70 mm to 110 mm, from 75 mm to 110 mm, from 80 mm to 110 mm, from 80 mm to 105 mm, or from 80 mm to 100 mm.

### Test Procedures

### Projection Height Measurement Method

Sample preparation: the height of the projections on the projection layer may be measured according to the following method. For articles such as diapers that are commonly packaged folded and packaged under compression, the articles to be tested are taken from the middle of the package and are carefully un-folded and placed on the frame as described below for a period of 2 hours before conducting the measurement. During this time, the samples are conditioned at about 23° C.±2° C. and about 50%±2% relative humidity.

Equipment: The projection heights are measured using a GFM MikroCAD Premium instrument commercially available from GFMesstechnik GmbH, Teltow/Berlin, Germany or similar equipment. The GFM MikroCAD Premium instrument includes the following main components: a) a DLP projector with direct digital controlled micro-mirrors; b) a CCD camera with at least a 1600×1200 pixel resolution; c) projection optics adapted to a measuring area of at least 60 mm×45 mm; d) recording optics adapted to a measuring area of at least 60 mm×45 mm; e) a table tripod based on a small hard stone plate; f) a blue LED light source; g) a measuring, control, and evaluation computer running ODSCAD software (version 6.2, or equivalent); and h) calibration plates for lateral (x-y) and vertical (z) calibration available from the vendor.

The GFM MikroCAD Premium system measures the surface height of a sample using the digital micro-mirror pattern fringe projection technique. The result of the analysis is a map of surface height (z-directional or z-axis) versus displacement in the x-y plane. The system has a field of view of 60×45 mm with an x-y pixel resolution of approximately 40 microns. The height resolution is set at 0.5 micron/count, with a height range of +/−15 mm. All testing is performed in a conditioned room maintained at about 23±2° C. and about 50±2% relative humidity.

A steel frame (100 mm square, 1.5 mm thick with an opening 70 mm square) is used to mount the specimen. Take the steel frame and place double-sided adhesive tape on the bottom surface surrounding the interior opening. To obtain a specimen, lay the absorbent article flat on a bench with the wearer-facing surface directed upward. Remove the release paper of the tape, and adhere the three-dimensional material to the steel frame of the absorbent article. Using a razor blade, excise the three-dimensional material from the underling layers of the absorbent article around the outer perimeter of the frame. Carefully remove the specimen such that its longitudinal and lateral extension is maintained. A cryogenic spray (such as Cyto-Freeze, Control Company, Houston, Tex.) can be used to remove the three-dimensional material specimen from the underling layers, if necessary. If the three-dimensional material cannot be easily removed from the topsheet, the measurements can be made with the three-dimensional material still attached to the topsheet. Five replicates obtained from five substantially similar absorbent articles are prepared for analysis.

Method steps: Calibrate the instrument according to manufacturer's specifications using the calibration plates for lateral (x-y axis) and vertical (z axis) available from the vendor.

Place the steel plate and specimen on the table beneath the camera, with the wearer-facing surface oriented toward the camera. Center the specimen within the camera field of view, so that only the specimen surface is visible in the image. Allow the specimen to lay flat with minimal wrinkles. The specimen is placed so that its middle is paced in the center of the field of the camera.

Collect a height image (z-direction) of the specimen by following the instrument manufacturer's recommended measurement procedures. Select the Technical Surface/Standard measurement program with the following operating parameters: Utilization of fast picture recording with a 3 frame delay. Dual phaseshifts are used with 1) 16 pixel stripe width with a picture count of 12 and 2) 32 pixel stripe width with a picture count of 8. A full Graycode starting with pixel 2 and ending with pixel 512. After selection of the measurement program, continue to follow the instrument manufacturer's recommended procedures for focusing the measurement system and performing the brightness adjustment. Perform the 3D measurement then save the height image and camera image files.

Load the height image into the analysis portion of the software via the clipboard. The following filtering procedure is then performed on each image: 1) removal of invalid points; 2) removal of peaks (small localized elevations); 3) polynomial filtering of the material part with a rank of n=5, with exclusion of 30% of the peaks and 30% of the valleys from the material part, and 5 cycles.

Projection Height: Draw a line connecting the peaks of a series of projections, with the line crossing a non-apertured land area located between each of the projections. Generate a sectional image of the height image along the drawn line. Along the sectional line, measure the vertical height (z-direction) difference between the peak of the projection and the adjacent valley of the land area. Record the height to the nearest 0.1 μm. Average together 10 different projection peak to land area height measures and report this value to the nearest 0.1 μm. This is the projection height.

### In-Bag Stack Height Test

The in-bag stack height of a package of absorbent articles is determined as follows:

Equipment: A thickness tester with a flat, rigid horizontal sliding plate is used. The thickness tester is configured so that the horizontal sliding plate moves freely in a vertical direction with the horizontal sliding plate always maintained in a horizontal orientation directly above a flat, rigid horizontal base plate. The thickness tester includes a suitable device for measuring the gap between the horizontal sliding plate and the horizontal base plate to within±0.5 mm. The horizontal sliding plate and the horizontal base plate are larger than the surface of the absorbent article package that contacts each plate, i.e. each plate extends past the contact surface of the absorbent article package in all directions. The horizontal sliding plate exerts a downward force of 850±1 gram-force (8.34 N) on the absorbent article package, which may be achieved by placing a suitable weight on the center of the non-package-contacting top surface of the horizontal sliding plate so that the total mass of the sliding plate plus added weight is 850±1 grams.

Test Procedure: Absorbent article packages are equilibrated at 23±2° C. and 50±5% relative humidity prior to measurement. The horizontal sliding plate is raised and an absorbent article package is placed centrally under the horizontal sliding plate in such a way that the absorbent articles within the package are in a horizontal orientation. Any handle or other packaging feature on the surfaces of the package that would contact either of the plates is folded flat against the surface of the package so as to minimize their impact on the measurement. The horizontal sliding plate is lowered slowly until it contacts the top surface of the package and then released. The gap between the horizontal plates is measured to within±0.5 mm ten seconds after releasing the horizontal sliding plate. Five identical packages (same size packages and same absorbent articles counts) are measured and the arithmetic mean is reported as the package width. The “In-Bag Stack Height”=(package width/absorbent article count per stack)×10 is calculated and reported to within±0.5 mm.

### Misc

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article having a wearer-facing side, a garment-facing side, a longitudinal axis extending from a front edge to a back edge and a transversal axis perpendicular to the longitudinal axis and extending from a first longitudinally-extending side edge to a second longitudinally-extending side edge; the absorbent article comprising:
a topsheet on the wearer-facing side;
a backsheet on the garment-facing side;
an absorbent core comprising an absorbent material, wherein the absorbent core comprises at least one longitudinally-extending channel substantially free of absorbent material and wherein the absorbent material comprises superabsorbent polymers;
a stretchable back waistband extending transversally adjacent the back edge of the article;
a pair of stretchable back ears each having a fastening tab, and
a landing zone on the garment-facing side of the article adjacent the front edge of the article, wherein the fastening tabs can be releasably engaged with the landing zone.

2. The absorbent article of claim 1, wherein the backsheet comprises a liquid impermeable film.

3. The absorbent article of claim 1, further comprising an acquisition layer between the topsheet and the absorbent core.

4. The absorbent article of claim 3, wherein the acquisition layer is an air-through bonded carded nonwoven.

5. The absorbent article of claim 1, wherein the absorbent material of the absorbent core comprises a mixture of fibers and superabsorbent polymers, with the absorbent material comprising at least about 55% superabsorbent polymers by weight of the absorbent material.

6. The absorbent article of claim 5, wherein the absorbent material comprises from about 60% to about 90% superabsorbent polymers by weight of the absorbent material.

7. The absorbent article of claim 1, wherein the absorbent material defines an absorbent material deposition area in the plane formed by the transversal axis and the longitudinal axis, wherein the absorbent material deposition area comprises a front edge, a back edge and two longitudinal side edges and is further shaped so that its width in the transversal direction varies along the position on the longitudinal axis.

8. The absorbent article of claim 7, wherein the width of the absorbent material area is maximum at the front edge and/or at the back edge of the absorbent material area, and is minimum at a longitudinal position between the front edge and the back edge of the absorbent material area.

9. The absorbent article of claim 1, wherein the absorbent core comprises a pair of channels disposed symmetrically relative to the longitudinal axis.

10. The absorbent article of claim 9, wherein these channels are straight and parallel to the longitudinal axis.

11. The absorbent article of claim 9, wherein these channels are inwardly curved towards the longitudinal axis so that the channels have a closest distance and a farthest distance measured in the transversal direction, wherein the closest distance is less than about 80% of the farthest distance.

12. The absorbent article of claim 1, wherein the at least one channel has at least partially a width of from about 2 mm to about 20 mm.

13. The absorbent article of claim 1, comprising a wetness indicator visible from the garment-facing side of the article wherein the wetness indicator is a composition comprising a hot-melt adhesive and a pH-indicator disposed between the absorbent core and the backsheet.

14. The absorbent article of claim 1, further comprising a three-dimensional material on the wearer-facing side, the three-dimensional material being joined to the topsheet, and comprising:
a. a fibrous support layer having an inner surface and an opposed outer surface;
b. a fibrous projection layer having an inner surface and an opposed outer surface, the outer surface of the support layer being in contact with the inner surface of the projection layer; and
c. a plurality of hollow projections formed from a first plurality of fibers in the projection layer, the hollow projections extending from the outer surface of the projection layer in a direction away from the support layer.

15. The absorbent article of claim 14, wherein the support layer and projection layer are substantially coterminous in the plane formed by the longitudinal axis and the transversal axis, and a second plurality of fibers are entangled at the interface of the support layer and the projection layer so that these layers form a unitary material.

16. An absorbent article having a wearer-facing side, a garment-facing side, a longitudinal axis extending from a front edge to a back edge and a transversal axis perpendicular to the longitudinal axis and extending from a first longitudinally-extending side edge to a second longitudinally-extending side edge; the absorbent article comprising:
a topsheet on the wearer-facing side;
a backsheet on the garment-facing side;
an absorbent core comprising an absorbent material, wherein the absorbent core comprises at least one longitudinally-extending channel substantially free of absorbent material;
a stretchable back waistband extending transversally adjacent the back edge of the article;
a pair of stretchable back ears each having a fastening tab, and
a landing zone on the garment-facing side of the article adjacent the front edge of the article, wherein the fastening tabs can be releasably engaged with the landing zone; and
wherein the absorbent core comprises a core wrap around the absorbent material, the core wrap comprising a top side and a bottom side, and the top side and the bottom side are bonded through the at least one channel.

17. The absorbent article of claim 16, wherein the top side and the bottom side of the core wrap are nonwovens which are non-adhesively bonded through the channels by at least one selected from ultrasonic bonding, pressure bonding and heat-bonding.

18. The absorbent article of claim 16, wherein the core wrap is formed by a single substrate which is C-wrapped around the absorbent material so that the bottom side or top side of the core wrap is formed by partially overlapping flaps of the substrate.

19. An absorbent article to claim 16, wherein the core wrap comprise a first substrate forming the top side or the bottom side of the core wrap and a second substrate at least partially forming the other side of the core wrap, wherein the first substrate is larger than the second substrate so that the first substrate can be folded over the second substrate to form a dual substrate C-wrap.

20. A package comprising a plurality of the absorbent articles, wherein the package has an In-Bag Stack Height of from about 70 mm to about 110 mm and wherein each absorbent article comprises:
a wearer-facing side, a garment-facing side, a longitudinal axis extending from a front edge to a back edge and a transversal axis perpendicular to the longitudinal axis and extending from a first longitudinally-extending side edge to a second longitudinally-extending side edge; the absorbent article comprising:
a topsheet on the wearer-facing side;
a backsheet on the garment-facing side;
an absorbent core comprising an absorbent material, wherein the absorbent core comprises at least one longitudinally-extending channel substantially free of absorbent material;
a stretchable back waistband extending transversally adjacent the back edge of the article;
a pair of stretchable back ears each having a fastening tab, and
a landing zone on the garment-facing side of the article adjacent the front edge of the article, wherein the fastening tabs can be releasably engaged with the landing zone.
